(19) United States
(12) Patent Application Publication
(10) Pub. No.: US 2014/0111773 A1
(43) Pub. Date: Apr. 24, 2014

(54) OPHTHALMOLOGIC APPARATUS, OPHTHALMOLOGIC CONTROL METHOD, AND PROGRAM

(71) Applicant: CANON KABUSHIKI KAISHA, Tokyo (JP)

(72) Inventor: Hiroshi Itoh, Yokohama-shi (JP)

(73) Assignee: CANON KABUSHIKI KAISHA, Tokyo (JP)

(21) Appl. No.: 14/055,093

(22) Filed: Oct. 16, 2013

(30) Foreign Application Priority Data
Oct. 18, 2012 (JP) 2012-230782

(51) Int. Cl. A61B 3/16; A61B 3/107

(52) U.S. Cl. CPC A61B 3/165; A61B 3/107
USPC 351/212

(57) ABSTRACT

An apparatus includes a position detecting unit for detecting the position when a positioning unit completes positioning in a working distance direction, a first acquisition unit for acquiring information about a curvature radius of a cornea of a subject's eye, a second acquisition unit for acquiring information about an intraocular pressure of the subject's eye at a predetermined position in a movable range, which is set in the working distance direction in such a way as not to exceed a proximity limit position relative to the subject's eye, and a control unit for controlling the second acquisition unit based on outputs of the position detecting unit and the first acquisition unit in such a way as to maintain a predetermined distance between the cornea of the subject's eye and the proximity limit position in the working distance direction, regardless of the curvature radius of the cornea of the subject's eye.

### BACKGROUND OF THE INVENTION

1. Field of the Invention

The present invention relates to an ophthalmologic apparatus that can measure an intraocular pressure of a subject's eye without directly contacting the subject's eye. Further, the present invention relates to an ophthalmologic control method and a related program.

2. Description of the Related Art

A refractometer/keratometer and a non-contact type tonometer are ophthalmologic apparatuses conventionally known. The refractometer/keratometer is generally used to measure the refractive power of a subject's eye or the shape of its cornea (i.e., the radius of curvature of the cornea). The non-contact type tonometer is generally used to measure the intraocular pressure with a spray of air. The refractometer/keratometer is conventionally provided as an integrated apparatus. On the other hand, the tonometer is conventionally provided as an independent apparatus because it is necessary to use an objective lens having an aperture to spray air to a subject. However, in an ophthalmologic office, reducing the space for an eye examination room or preventing a patient from being forced to move or walk for eye examination is generally desired. An ophthalmologic apparatus having been recently proposed to satisfy such requirements includes a refractometer/keratometer and a tonometer that are integrally accommodated in a single casing.

As discussed in Japanese Patent Application Laid-Open No. 2007-282672, there is a conventional ophthalmologic apparatus that is first functionally operable as a refractometer/keratometer to measure the refractive power of a subject's eye or the radius of curvature of its cornea and is subsequently operable as a tonometer to measure the intraocular pressure of the subject's eye. However, the refractometer/keratometer and the tonometer are different from each other in the distance (i.e., working distance) between the apparatus (i.e., the objective lens) and the cornea of the subject's eye. More specifically, the working distance of the refractometer/keratometer is 30 mm to 40 mm. The working distance of the tonometer is appropriately 11 mm.

Therefore, especially, in the measurement using the tonometer whose working distance is relatively short, it is necessary for an operator to prevent an intraocular pressure measurement nozzle from contacting the cornea of a subject's eye. In this respect, the ophthalmologic apparatus discussed in Japanese Patent Application Laid-Open No. 2007-282672 determines a proximity limit in such a way as to prevent the intraocular pressure measurement nozzle from approaching the subject's eye beyond a proximity limit position. More specifically, the apparatus acquires information about the position of a measurement unit body in a back-and-forth direction when the apparatus performs an eye refractivity measurement and then determines the proximity limit position to be referred to in an intraocular pressure measurement based on the acquired positional information.

Further, as discussed in Japanese Patent Application Laid-Open No. 2000-70224, there is a conventional ophthalmologic apparatus that is first functionally operable as a keratometer that measures the radius of curvature of the cornea of a subject's eye and is subsequently operable as a tonometer that measures the intraocular pressure of the subject's eye. Further, to prevent the radius of curvature of the cornea from causing a positioning error, the apparatus starts measuring the intraocular pressure after completing the positioning for maintaining a constant distance between an intraocular pressure measurement nozzle and a corneal surface (i.e., a clearance in a working distance direction). However, according to Japanese Patent Application Laid-Open No. 2007-282672, the apparatus adjusts the distance between the proximity limit position for the intraocular pressure measurement nozzle and the subject's eye in such a way as to maintain a constant distance between the proximity limit position and a corneal focal position where a virtual image of an alignment index can be formed (i.e., a position away from the apex of the cornea by an amount equivalent to a half of the radius of curvature of the cornea). Therefore, the distance from the apex of the cornea to the proximity limit position causes a deviation depending on the radius of curvature of the cornea. Thus, the subject feels that something is wrong when the apparatus is approaching the subject's eye, in particular, when the subject's eye has a larger radius of curvature. The subject may suddenly move the eye due to the feeling of wrongness. Therefore, the positioning operation becomes difficult or becomes unstable and takes a long time to acquire information about the intraocular pressure.

The ophthalmologic apparatus discussed in Japanese Patent Application Laid-Open No. 2000-70224 performs positioning for maintaining a constant distance between the corneal surface and the intraocular pressure measurement nozzle, before starting the measurement, irrespective of the radius of curvature of the cornea. However, there is not any restriction with respect to a movable range of the nozzle (including the position of the intraocular pressure measurement nozzle in the measurement) before starting the measurement. Therefore, a problem similar to that in Japanese Patent Application Laid-Open No. 2007-282672 occurs when an operator adjusts the nozzle position in the working distance direction before starting the measurement especially when the subject's eye has a larger radius of curvature.

### SUMMARY OF THE INVENTION

The present invention is directed to an ophthalmologic apparatus, an ophthalmologic control method, and a related program, in which the proximity limit position for a unit configured to acquire information about the intraocular pressure of a subject's eye can be a constant distance away from a cornea of the subject's eye, regardless of the radius of curvature of the cornea of the subject's eye.

According to an aspect of the present invention, an ophthalmologic apparatus includes a positioning unit configured to perform positioning for a subject's eye and an apparatus body by projecting an index light flux on a cornea of the subject's eye and capturing a corneal reflection image with light reflected from the cornea of the subject's eye, a position detecting unit configured to detect the position of the apparatus body when the positioning unit completes the positioning in a working distance direction, a first acquisition unit provided on the apparatus body and configured to acquire information about a radius of curvature of the cornea of the subject's eye, a second acquisition unit provided on the apparatus body and configured to acquire information about an intraocular pressure of the subject's eye at a predetermined position in a movable range, which is set in the working distance direction in such a way as not to exceed a proximity limit position relative to the subject's eye, and a control unit configured to control the second acquisition unit based on outputs of the position detecting unit and the first acquisition unit in such a way as to maintain a predetermined distance between the cornea of the subject's eye and the proximity limit position for the second acquisition unit in the working distance direction, regardless of the radius of curvature of the cornea of the subject's eye.

According to another aspect of the present invention, an ophthalmologic control method includes performing positioning for a subject's eye and an apparatus body by projecting an index light flux on a cornea of the subject's eye and capturing a corneal reflection image with light reflected from the cornea of the subject's eye, detecting the position of the apparatus body when the positioning in a working distance direction is completed, causing a first acquisition unit provided on the apparatus body to acquire information about a radius of curvature of the cornea of the subject's eye, controlling a second acquisition unit provided on the apparatus body and configured to acquire information about an intraocular pressure of the subject's eye based on the detected position of the apparatus body and the acquired information about the radius of curvature of the cornea, in such a way as to set the proximity limit position in relation to the subject's eye to be a predetermined distance away from the cornea of the subject's eye in the working distance direction, regardless of the radius of curvature of the cornea of the subject's eye, and causing the second acquisition unit to acquire information about the intraocular pressure of the subject's eye at a predetermined position in a movable range, which is set in the working distance direction in such a way as not to exceed the proximity limit position.

According to an exemplary embodiment of the present invention, it is feasible to maintain a constant distance between the cornea of a subject's eye and the proximity limit position for a unit configured to acquire information about the intraocular pressure of the subject's eye, irrespective of the radius of curvature of the cornea of the subject's eye. Therefore, it is feasible to prevent the subject from feeling that something is wrong when the apparatus is approaching the subject's eye even if the subject's eye has a larger radius of curvature. Further, an operator can easily perform a positioning operation because the subject does not move the eye due to the feeling of wrongness. The operator can quickly and stably acquire the information about the intraocular pressure.

Further features the present invention will become apparent from the following description of exemplary embodiments with reference to the attached drawings.

### BRIEF DESCRIPTION OF THE DRAWINGS

FIG. 1A schematically illustrates an ophthalmologic apparatus according to an exemplary embodiment of the present invention, and FIG. 1B illustrates a proximity limit position Dlimit of a stage in an intraocular pressure measurement according to an exemplary embodiment of the present invention.

FIG. 2 illustrates a measurement unit according to an exemplary embodiment of the present invention.

FIG. 3 is a perspective view illustrating an alignment prism diaphragm according to an exemplary embodiment of the present invention.

FIG. 4A illustrates an anterior eye observation image including alignment bright spots according to an exemplary embodiment of the present invention, and FIG. 4B is a partially enlarged view of FIG. 4A.

FIGS. 5A, 5B, 5C, and 5D relate to corneal reflection images of index light fluxes used for positioning, in which FIG. 5A illustrates a state where the positioning in the right-and-left direction is bad, FIG. 5B illustrates a state where the positioning in the up-and-down direction is bad, FIG. 5C illustrates a state where the positioning in the back-and-forth direction is bad, and FIG. 5D illustrates a state where the positioning is good.

FIG. 6A illustrates a state where the back-and-forth alignment using the alignment prism diaphragm is good, FIG. 6B illustrates a state where the alignment is too far, and FIG. 6C illustrates a state where the alignment is too close.

FIG. 7 is a flowchart illustrating a measurement operation according to an exemplary embodiment of the present invention.

FIG. 8A illustrates proximity limit positions displayed on a display unit according to an exemplary embodiment of the present invention, and FIG. 8B is a partially enlarged view of FIG. 8A.

### DESCRIPTION OF THE EMBODIMENTS

Various exemplary embodiments, features, and aspects of the invention will be described in detail below with reference to the drawings.

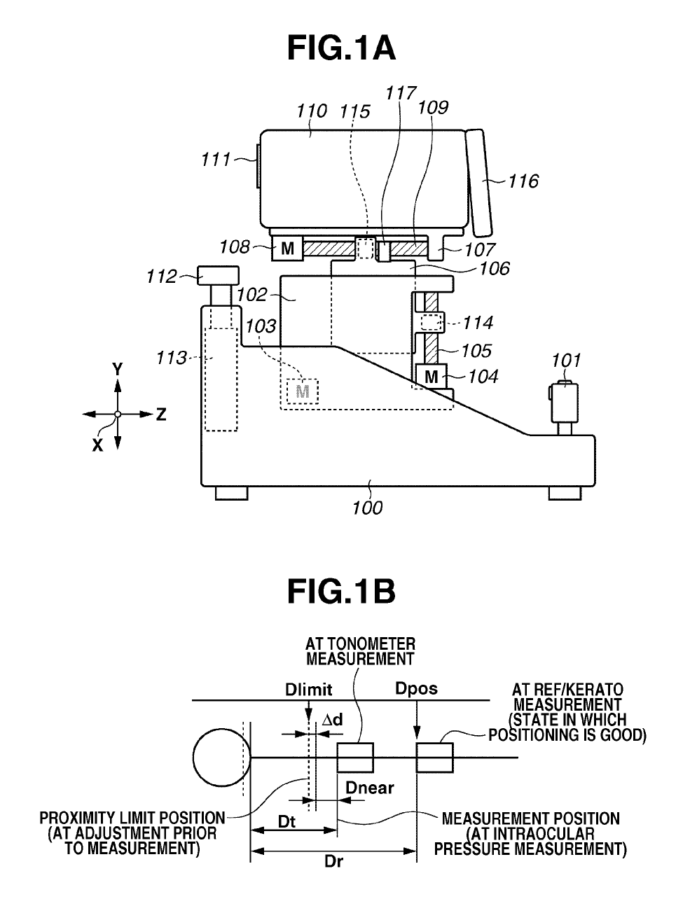

FIG. 1A illustrates an example configuration of an ophthalmologic apparatus that accommodates an optical system including two measurement units, which are provided in a measurement unit 110 (i.e., an apparatus body). A measurement unit A is a refractometer/keratometer that is functionally operable as a first acquisition unit. A measurement unit B is a tonometer that is functionally operable as a second acquisition unit. A frame 102 is movable relative to a base 100 in the right-and-left direction (hereinafter, referred to as “X-axis direction”). A driving mechanism operable in the X-axis direction includes an X-axis driving motor 103 fixed to the base 100, a feed screw (not illustrated) connected to an output shaft of the motor 103, and a nut (not illustrated) that is movable along the feed screw in the X-axis direction and is fixed to the frame 102. When a rotational force of the motor 103 is transmitted to the frame 102 via the feed screw and the nut, the frame 102 can move in the X-axis direction.

A frame 106 is movable relative to the frame 102 in an up-and-down direction (hereinafter, referred to “Y-axis direction”). A driving mechanism operable in the Y-axis direction includes a Y-axis driving motor 104 fixed to the frame 102, a feed screw 105 connected to an output shaft of the motor 104, and a nut 114 that is movable along the feed screw in the Y-axis direction and is fixed to the frame 106. When a rotational force of the motor 104 is transmitted to the frame 106 via the feed screw and the nut, the frame 106 can move in the Y-axis direction.

A frame 107 is movable relative to the frame 106 in a back-and-forth direction (hereinafter, referred to as “Z-axis direction”). A driving mechanism operable in the Z-axis direction includes a Z-axis driving motor 108 fixed to the frame 107, a feed screw 109 connected to an output shaft of the motor 108, and a nut 115 that is movable along the feed screw in the Z-axis direction and is fixed to the frame 106. Further, a position detecting unit 117 is provided to detect the position of the measurement unit in the Z-axis direction.

When a rotational force of the motor 108 is transmitted to the frame 107 via the feed screw 109 and the nut, the frame 107 can move in the Z-axis direction. The measurement unit 110, which performs a measurement operation, is fixed on the frame 107. A light source (not illustrated) usable to perform positioning (alignment) for a subject's eye and a kerato light source unit 111 usable to measure the curvature of a cornea are provided on a subject side end portion of the measurement unit 110.

Further, a joystick 101 is provided on the frame 100. The joystick 101 (i.e., an operation member) is operable to perform positioning for the measurement unit 110 relative to a subject's eye E. An operator can tilt the joystick 101 during a measurement operation to perform positional adjustment.

In a measurement of eye refractivity and corneal curvature, a subject places a subject's jaw on a jaw receiver 112 and presses a subject's forehead against a forehead receiving portion of a face receiving frame (not illustrated) fixed to the frame 100 so that the position of the subject's eye can be fixed. Further, a jaw receiver driving mechanism 113 is provided to adjust the position of the jaw receiver 112 in the Y-axis direction according to a face size of the subject.

A liquid crystal display (LCD) monitor 116 is provided on an operator side end portion of the measurement unit 110. The LCD monitor 116 is a display member that enables an operator to observe the subject's eye E. A measurement result can be displayed on the LCD monitor 116.

(Refractometer/Keratometer Serving as Measurement Unit A)

### (Refractometer)

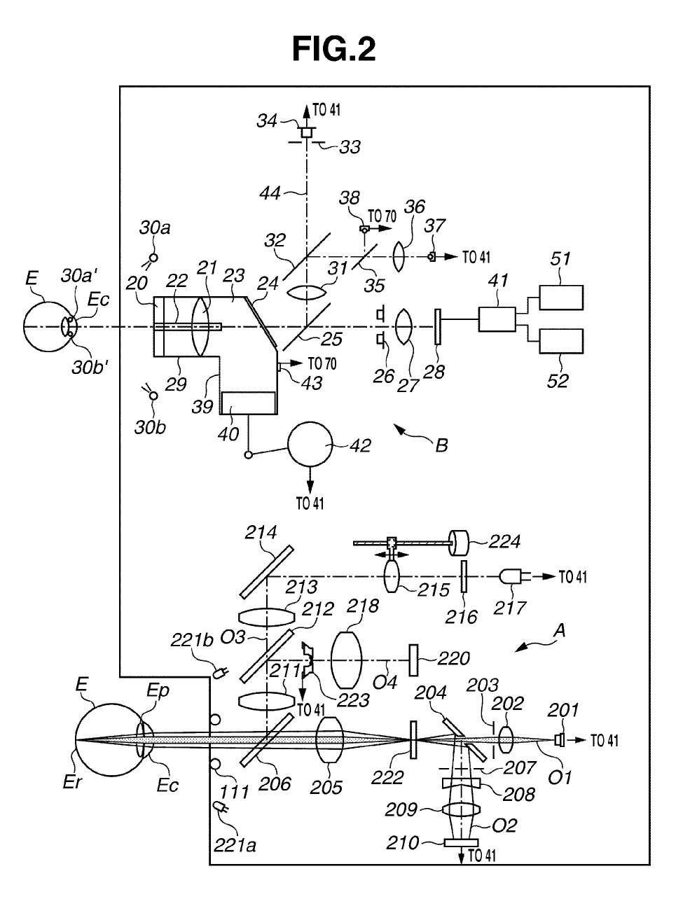

FIG. 2 illustrates the optical system provided in the measurement unit 110. First, the measurement unit A is a refractometer that is operable as an eye refractivity meter that can acquire refractive power information. The measurement unit A includes a measurement light source 201 dedicated to eye refractivity measurement, which can emit light having a wavelength of 880 nm. Further, the measurement unit A includes a lens 202, a diaphragm 203 that is substantially conjugate with a pupil Ep of the subject's eye E, a perforated mirror 204, and a lens 205 that are successively arrayed on an optical path 01 extending from the measurement light source 201 to the subject's eye E.

Further, a dichroic mirror 206 is disposed next to the lens 205 on the side near the subject's eye E. The dichroic mirror 206 totally reflects the infrared and visible light having a wavelength equal to or less than 880 nm to partly reflect the light flux having a wavelength equal to or greater than 880 nm. A diaphragm 207 having a ring-shaped slit, a light flux splitting prism 208, a lens 209, and an image sensor 210 are successively arrayed on an optical path 02 extending in the reflection direction of the perforated mirror 204. The diaphragm 207 is substantially conjugate with the pupil Ep.

The above-mentioned optical system is usable for eye refractivity measurement. The diaphragm 203 restricts the light flux emitted from the measurement light source 201. The lens 202 forms a primary image on the foreside of the lens 205. Then, the light flux penetrates the lens 205 and the dichroic mirror 206 and reaches the center of the pupil of the subject's eye E. Then, a fundus Er of the subject's eye E reflects the light flux. The light reflected on the fundus Er passes through the center of the pupil and reaches the lens 205 again. The light flux penetrates the lens 205. Then, the perforated mirror 204 reflects the light flux at a peripheral region around a central hole.

The light flux reflected by the perforated mirror 204 is pupil-separated by the diaphragm 207 (i.e., the element substantially conjugate with the pupil Ep of the subject's eye) and the light flux splitting prism 208. Then, the light flux is projected as a ring image on a light-receiving surface of the image sensor 210. If the subject's eye E is emmetropia, the projected ring image becomes a predetermined circle. If the subject's eye E is myopia, the projected ring image becomes a smaller circle compared to that of the emmetropic eye. If the subject's eye E is hypermetropia, the projected ring image becomes a larger circle compared to that of the emmetropic eye. If the subject's eye E is astigmatism, the projected ring image becomes elliptic. An angle between the horizontal axis and the ellipse can be defined as an astigmatic axis angle. A factor derived from the ellipse is usable in obtaining the refractive power.

On the other hand, a fixation target projection optical system and an alignment light-receiving optical system are disposed in the reflection direction of the dichroic mirror 206. The alignment light-receiving optical system is commonly usable to observe an anterior eye portion of the subject's eye and detect the alignment. A lens 211, a dichroic mirror 212, a lens 213, a reflecting mirror 214, a lens 215, a fixation target 216, and a fixation target illumination light source 217 are successively arrayed on an optical path 03 of the fixation target projection optical system.

When the fixation target illumination light source 217 is activated for fixation guidance, the projection light flux of the light source 217 illuminates a back surface of the fixation target 216 and reaches the fundus Er of the subject's eye E via the lens 215, the reflection mirror 214, the lens 213, the dichroic mirror 212, and the lens 211. A fixation guidance motor 224 can move the lens 215 in the optical axis direction to perform visual degree guidance for the subject's eye E and realize a fogging state.

(Keratometer)

In FIG. 2, a ring-shaped Kerato measurement light source 111 is disposed around the optical axis, so that a ring-shaped light flux can be projected on a cornea of the subject's eye E. The light flux reflected on the cornea of the subject's eye E can be projected as a ring image on a light-receiving surface of the image sensor 220 via the lenses 211 and 218. If the cornea of the subject's eye E has a standard shape, the projected ring image becomes a standard circle having a predetermined size. If the subject's eye E has a smaller radius of curvature, the projected ring image becomes a circle that is smaller than the standard circle. If the subject's eye E has a larger radius of curvature, the projected ring image becomes a circle that is larger than the standard circle. If the subject's eye E is astigmatism, the projected ring image becomes elliptic. An angle between the horizontal axis and the ellipse can be defined as the astigmatic axis angle. A factor derived from the ellipse is usable in obtaining the shape of the cornea.

(Positioning)

Further, an alignment prism diaphragm 223, the lens 218, and the image sensor 220 are successively arrayed on an optical path 04 extending in the reflection direction of the dichroic mirror 212. The alignment prism diaphragm 223 can be inserted and removed by an alignment prism diaphragm insertion and removal solenoid (not illustrated). When the alignment prism diaphragm 223 is inserted, the alignment prism diaphragm 223 can be positioned on the optical path 04 to perform alignment. When the alignment prism diaphragm 223 is removed, the alignment prism diaphragm 223 is not present on the optical path to perform anterior eye observation or transillumination observation.

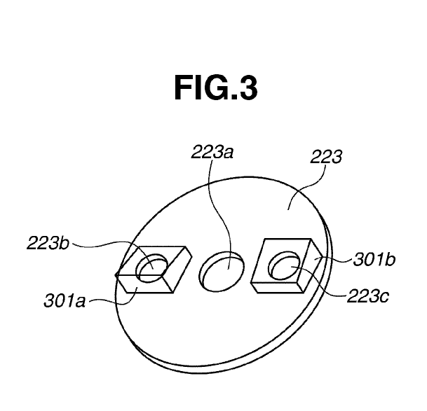

As illustrated in FIG. 3, the alignment prism diaphragm 223 includes a disk-shaped diaphragm plate, which has three apertures (e.g., a central aperture 223a, and two apertures 223b and 223c provided at both ends of the plate in the right-and-left direction). Further, alignment prisms 301a and 301b are attached to a surface of the plate opposed to the dichroic mirror 212 in such a way as to cover the apertures 223b and 223c provided at both ends in the right-and-left direction, respectively. The light flux, if its wavelength is in the vicinity of 880 nm, can penetrate the alignment prisms 301a and 301b.

Further, two light sources 221a and 221b dedicated to anterior eye illumination are disposed in a diagonally forward direction in relation to the anterior eye portion of the subject's eye E. For example, the light sources 221a and 221b can emit light having a wavelength of approximately 780 nm. The light flux from the anterior eye portion of the subject's eye E, which is emitted from the anterior eye illumination light sources 221a and 221b, passes through the dichroic mirror 206, the lens 211, the dichroic mirror 212, and the central aperture 223a of the alignment prism diaphragm 223 and forms an image on a light-receiving sensor surface of the image sensor 220. The light flux having a wavelength of 780 nm or more, emitted from the anterior eye illumination light sources 221a and 221b, can pass through the central aperture 223a of the alignment prism diaphragm 223.

The measurement light source 201 dedicated to the eye refractivity measurement can be commonly used for the alignment detection. A diffusion plate insertion and removal solenoid (not illustrated) can move a semi-transparent diffusion plate 222 so that the semi-transparent diffusion plate 222 can be placed on the optical path in an alignment operation. The insertion position of the diffusion plate 222 is a primary image-forming position, at which the projection lens 202 primarily forms an image of the light emitted from the measurement light source 201, and is the focal position of the lens 205. Thus, the image of the light emitted from the measurement light source 201 is once formed on the diffusion plate 222. The image formed on the diffusion plate 222 serves as a secondary light source. The light flux from the diffusion plate 222 travels as a thick parallel light flux via the lens 205 toward the subject's eye E.

The cornea Ef of the subject's eye E reflects the parallel light flux. The reflected light flux forms a bright spot image as an index image. Then, the dichroic mirror 206 partly reflects the light flux again. The dichroic mirror 212 reflects the light flux reaching from the dichroic mirror 206 via the lens 211. The light flux reflected by the dichroic mirror 212 penetrates the alignment prism diaphragm 223. Then, the lens 218 converges the light flux and forms an image on the image sensor 220.

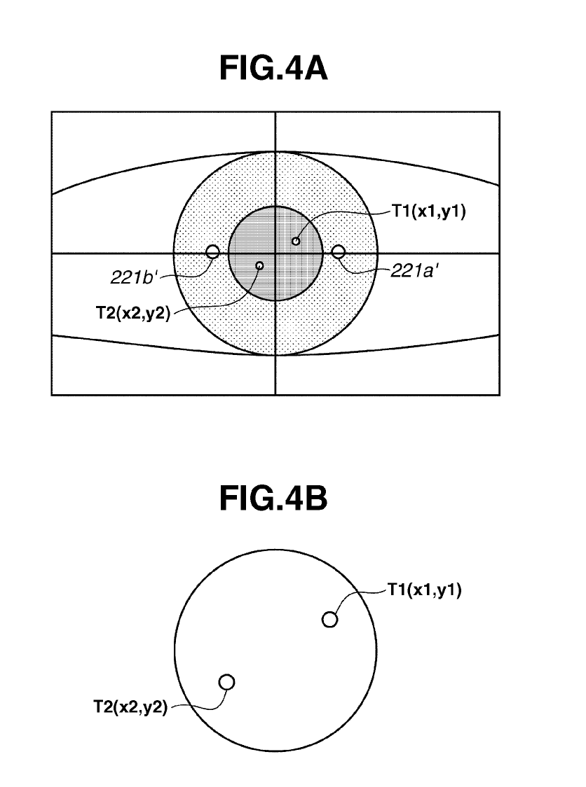

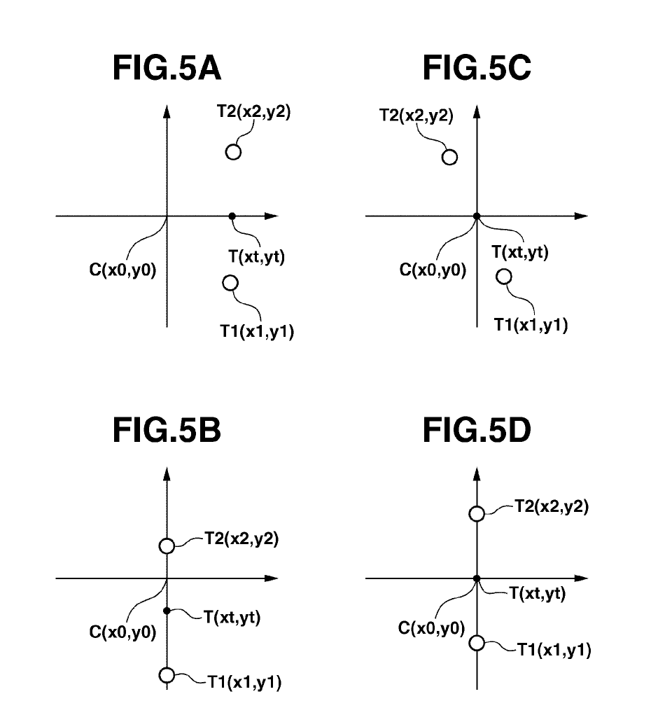

More specifically, as illustrated in FIGS. 4A and 4B and in FIGS. 5A, 5B, 5C, and 5D, the apertures 223a, 223b, and 223c and the prisms 301a and 301b of the alignment prism diaphragm 223 cooperatively divide the light flux and form index images T1, T2, and T on the image sensor 220. Although not illustrated in FIGS. 4A and 4B, the index image T is formed at an intermediate position between the index images T1 and T2. Further, the image sensor 220 captures an image of bright spot images 30a′ and 30b′ of the external eye illumination light sources 221a and 221b together with the anterior eye portion of the subject's eye E illuminated by the external eye illumination light sources 221a and 221b.

The light flux having a wavelength of 780 nm or more, which is emitted from the anterior eye illumination light sources 221a and 221b, can pass through the central aperture 223a of the alignment prism diaphragm 223. Therefore, the light flux reflected on the anterior eye portion illuminated by the anterior eye illumination light sources 221a and 221b travels along a path of the observation optical system, similar to the light flux reflected on the cornea Ef. The light flux passes through the aperture 223a of the alignment prism diaphragm 223. The image-forming lens 218 forms an image on the image sensor 220.

The light flux having penetrated through each of the alignment prisms 301a and 301b deflects in the up-and-down direction. The alignment for the subject's eye E can be performed based on a positional relationship between light fluxes passing through these prisms. FIG. 5A illustrates a case where the positioning in the right-and-left direction is bad. FIG. 5B illustrates a case where the positioning in the up-and-down direction is bad. FIG. 5C illustrates a case where the positioning in the back-and-forth direction is bad. FIG. 5D illustrates a case where the positioning is good.

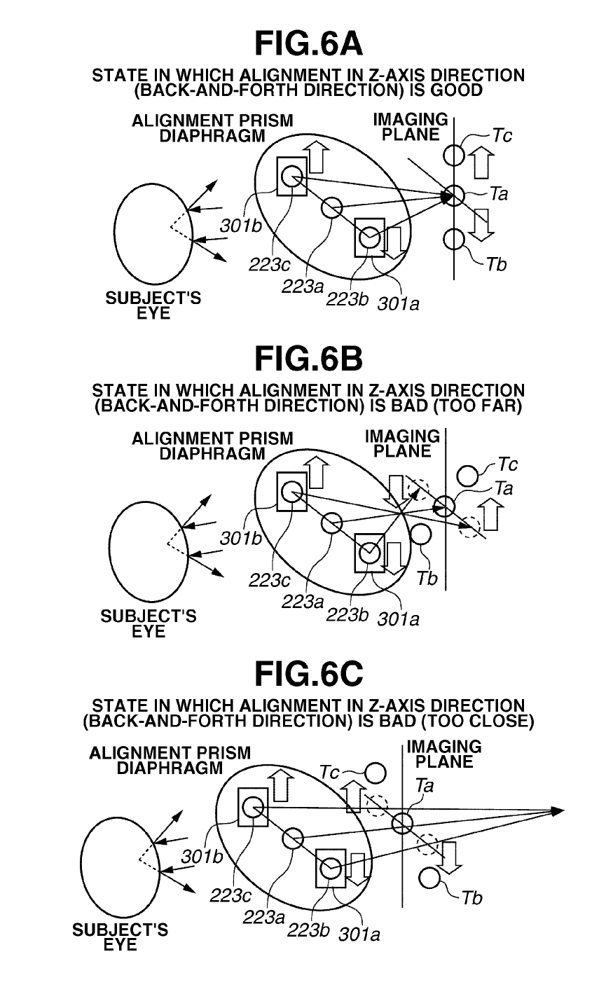

Further, FIG. 6A illustrates a case where the positioning (alignment) in the Z-axis direction (i.e., the back-and-forth direction) is good, in which three corneal bright spots Tc (corresponding to T1), Tb (corresponding to T2), and Ta (corresponding to T) are arrayed straight in a direction perpendicular to the horizontal direction. FIG. 6B illustrates a case where the positioning (alignment) in the Z-axis direction (i.e., the back-and-forth direction) is bad (too far). FIG. 6C illustrates a case where the positioning (alignment) in the Z-axis direction (i.e., the back-and-forth direction) is bad (too close).

(Tonometer serving as the measurement unit B) Next, the measurement unit B of the tonometer is described in detail below. A nozzle 22 is disposed on the common central axis of a parallel plane glass 20 and an objective lens 21 in such a way as to be opposed to a cornea Ec of the subject's eye E (although the subject's eye E cannot be simultaneously present at the measurement units A and B). Further, an air chamber 23, an observation window 24, a dichroic mirror 25, a prism diaphragm 26, an image-forming lens 27, and an image sensor 28 are successively arrayed on the behind side of the nozzle 22 along a straight line, which serves as a light-receiving optical path and an alignment detection optical path of an optical system that observes the subject's eye E. The prism diaphragm 26 is functionally comparable to the alignment prism diaphragm 223.

The parallel plane glass 20 and the objective lens 21 are supported by an objective lens barrel 29. Two external eye illumination light sources 30a and 30b, each illuminating the subject's eye E, are disposed on the outside of the parallel plane glass 20. Although the external eye illumination light sources 30a and 30b are located at upper and lower positions in the drawing, the actual layout of these light sources 30a and 30b is vertical to the illustrated direction in such a way to be opposed to the optical axis.

A relay lens 31, a half mirror 32, an aperture 33, and a light-sensitive element 34 are disposed in the reflection direction of the dichroic mirror 25. The setup position of the aperture 33 is a position where a corneal reflection image formed by a measurement light source 37 becomes conjugate when a predetermined deformation occurs. The aperture 33 and the light-sensitive element 34 cooperatively constitute a deformation detection light-receiving optical system 44 configured to detect a deformation of the cornea Ec occurring in the visual axis direction. The relay lens 31 is designed in such a way to form a corneal reflection image having a size substantially similar to the aperture 33 when the cornea Ec causes the predetermined deformation.

A half mirror 35, a projection lens 36, and the measurement light source 37 are disposed in the incident direction of the half mirror 32. The measurement light source 37 is constituted by a near infrared light-emitting diode (LED) that can emit light having an invisible wavelength usable for measurement and alignment for the subject's eye E. Further, a fixation light source 38, which is constituted by an LED fixated by the subject, is disposed in the incident direction of the half mirror 35.

A piston 40 is fitted to a cylinder 39 (i.e., a part of the air chamber 23). The piston 40 can be driven by a solenoid 42. A pressure sensor 43, which is configured to monitor the inner pressure, is disposed in the air chamber 23. Measuring an intraocular pressure of the subject's eye E is feasible by obtaining an output value of the pressure sensor 43 when the cornea Ec causes the above-mentioned predetermined deformation.

A control unit 41 is connected to a storage unit 51 and an arithmetic unit 52. The storage unit 51 stores information about working distance Dr (e.g., 35 mm) in the Ref/Kerato measurement (i.e., eye refractivity/cornea shape measurement) illustrated in FIG. 1B and working distance Dt (e.g., 11 mm) in the intraocular pressure measurement. Further, the storage unit 51 stores information about proximity limit distance Dnear (e.g., 5 mm) that represents the distance from the cornea of the subject's eye E to the measurement unit 110 (including the nozzle 22) positioned closest to the subject's eye E in the intraocular pressure measurement. The proximity limit distance corresponds to the distance between the cornea of the subject's eye E and the front end (i.e., a subject's eye side) of the nozzle 22 positioned closest to the subject's eye E.

Further, the storage unit 51 stores a table (see table 1) in which the radius of curvature of the cornea of the subject's eye E is associated with a correction amount for the proximity limit position in the working distance direction. Further, the storage unit 51 stores information about stage position Dpos that represents the position of the measurement unit 110 (i.e., the apparatus body in the Ref/Kerato measurement) read by the position detecting unit 117 when the positioning in the working distance direction (i.e., the Z-axis direction indicating the back-and-forth direction) is performed.

TABLE 1

| Radius of curvature of cornea | | | | | | | |
|---|---|---|---|---|---|---|---|
| | 5 | 6 | 7 | 7.8 | 8 | 9 | 10 |
| ΔD | 1.4 | 0.9 | 0.4 | 0 | −0.1 | −0.6 | −1.1 |

(Flowchart)

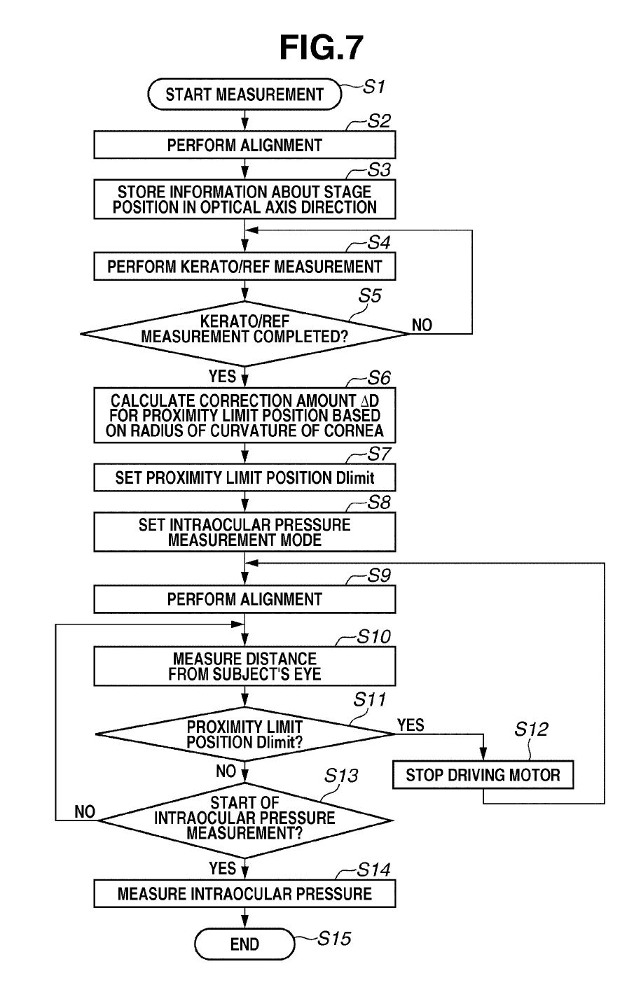

Hereinafter, an example measurement that can be performed by the ophthalmologic apparatus according to the present exemplary embodiment is described in detail below with reference to a flowchart illustrated in FIG. 7. The measurement performed in this case is a refractometer/keratometer measurement characterized in that the working distance is long. First, in step S1, the measurement unit A is opposed to the subject's eye E. In step S2, an operator operates the joystick 101 to perform positioning (alignment) in such a way that two index images T1 and T2 are arrayed straight at the central portion of the screen.

In the present exemplary embodiment, the operator performs the positioning process (i.e., operation in step S2) in such a way as to equalize a corneal focal position where a virtual image of an alignment index is formed (i.e., a position spaced from the apex of the cornea by an amount equivalent to a half of the radius of curvature of the cornea) with the central portion of the image sensor 220.

The movement of two index images T1 and T2 in an alignment operation is described in detail below with reference to FIGS. 5A to 5D. In FIGS. 5A to 5D, a point C(x0, y0) where the x-axis and the y-axis intersect with each other represents the center of the cornea. T1(x1, y1) and T2(x2, y2) represent coordinate values of two index images T1 and T2. T(xt, yt) represents coordinate values of a midpoint between two index images T1 and T2.

If the central axis of the lens 205 deviates from the center of the cornea in the right-and-left direction, x1 coincides with x2 as illustrated in FIG. 5A. Coordinate values y0 and yt in the y-direction coincide with each other with respect to the center C(x0, y0) of the cornea. However, xt is different from x0. Therefore, the operator moves the measurement unit in the right-and-left direction in such away as to equalize xt with x0. Similarly, the central axis of the lens 205 deviates from the center of the cornea in the up-and-down direction, x1 coincides with x2 as illustrated in FIG. 5B. In this case, yt is different from y0. Therefore, the operator operates the joystick 101 to move the measurement unit in the up-and-down direction to equalize yt with y0.

If the central axis of the lens 205 deviates from the center of the cornea in the working distance direction (i.e., the Z-axis direction), the centroid position coincides with the center of the cornea as illustrated in FIG. 5C. However, x1 is different from x2 and y1 is different from y2. Therefore, the operator moves the measurement unit in the central axis direction of the lens 205 to equalize the y-coordinate values y1 and y2. However, the positioning index images T1 and T2 are virtual images (i.e., corneal focal positions) formed by the cornea of the subject's eye E. Therefore, the distance from the corneal focal point to the apex of the cornea is variable depending on the radius of curvature of the cornea of the subject's eye E. The working distance from the apex of the cornea of the measurement unit 110 is variable depending on the radius of curvature of the cornea of the subject's eye E.

The operator operates the joystick 101 to perform the alignment to obtain an index image illustrated in FIG. 5D (see step S2). Then, in step S3, namely after completing the alignment, the operator presses a measurement switch provided on the joystick 101 to start the Ref/Kerato measurement. The control unit 41 causes the position detecting unit 117 to read the present position of the measurement unit A in the Z-axis direction and stores the read positional data in the storage unit 51. More specifically, in step S3 (i.e., in an example position detecting process), the position detecting unit 117 (see FIG. 1) detects the position of the measurement unit 110 based on an alignment index image (whose virtual image is formed at the corneal focal position) in a state where the alignment has been completed.

Next, the operation proceeds to step S4, in which the operator starts the Kerato measurement. The operator activates the Kerato measurement light source 111 to irradiate the subject's eye E with a light flux and removes the alignment prism diaphragm 223 from the optical path. Then, the operator analyzes the ring image captured by the imaging unit 220 to calculate the radius of curvature of the cornea. After performing the above-mentioned measurement a predetermined number of times, the operator deactivates the Kerato measurement light source 111 and starts the Ref measurement. More specifically, the operator activates the measurement light source 201 and analyzes a ring image captured by the image sensor 210 to calculate a Ref value (i.e., eye refractivity). Next, in step S5, it is determined whether the above-mentioned Kerato/Ref measurement has been performed a predetermined number of times. More specifically, the radius of curvature of the cornea of the subject's eye E and the eye refractivity can be acquired in steps S4 and S5 (i.e., example ophthalmologic information acquisition process).

If it is determined that the above-mentioned Kerato/Ref measurement has been performed the predetermined number of times (YES in step S5), then in step S6, the apparatus determines a correction amount AD with reference to a measurement result of the radius of curvature of the cornea and the table 1 stored in the storage unit 51, in such a way as to set the proximity limit distance in the intraocular pressure measurement to be a constant value, regardless of the radius of curvature of the cornea of the subject's eye E. The correction amount ΔD is a half of the difference between the curvature radius of the subject's eye E and the curvature radius of a standard subject's eye. Then, in step S7, the arithmetic unit 52 calculates the proximity limit position Dlimit for the measurement unit 110 in the intraocular pressure measurement according to the formula Dlimit=Dpos−(Dr−Dt+Dnear+ΔD), as illustrated in FIG. 1C. The control unit 41 sets the calculated proximity limit position Dlimit.

In the above-mentioned formula, Dr represents the working distance of the measurement unit 110 from the apex of the cornea in the Ref/Kerato measurement, Dt represents the working distance of the measurement unit 110 from the apex of the cornea in the intraocular pressure measurement, and Dnear represents the proximity limit distance in the intraocular pressure measurement as a standard proximity distance for the standard subject's eye. Dr−Dt+Dnear=Do is a constant value that can be determined as a setting value. Thus, the above-mentioned formula can be rewritten into Dlimit=Dpos−(Do+ΔD). More specifically, a distance obtainable by adding a half of the difference between the curvature radius of the subject's eye E and the curvature radius of the standard subject's eye, as the correction amount ΔD, to Do that corresponds to the standard proximity distance Dnear determined beforehand for the standard subject's eye is determined as a proximity distance that corresponds to the proximity limit position relative to the subject's eye E.

As an example, for example, when the stage position (i.e., the measurement unit 110) read by the position detecting unit 117 is spaced from a reference position by 50 mm (a positive value indicates the direction departing from the subject's eye), and if the radius of curvature of the cornea of the subject's eye is 9 mm, the correction value ΔD defined in the table 1 is 0.6. Therefore, the proximity limit position Dlimit is set to 21.6 mm (i.e., a position calculated according to Dlimit=50−(35−11+5−0.6)).

As described above, it is feasible to set the proximity limit distance to be constant in the intraocular pressure measurement regardless of the radius of curvature of the cornea of the subject's eye by correcting the proximity limit position with reference to the radius of curvature of the cornea. In the table 1, the radius of curvature of the cornea takes discrete values changing in increments of 1 mm. However, the amount to be incremented can be set to a smaller value. Alternatively, it is useful to calculate a correction value by interpolating the stored values.

In step S8, a measurement unit switching unit (not illustrated) causes the control unit 41 to switch the measurement unit A to the measurement unit B and to bring the operational mode into an intraocular pressure measurement mode. Then, in step S9, the operator operates the joystick 101 to perform alignment in the same manner as the processing performed in step S2. More specifically, in step S9 (i.e., an example positioning process), the prism diaphragm 26 (i.e., an element functionally similar to the alignment prism diaphragm 223) forms an image of the corneal focal position (i.e., the position where a virtual image of the alignment index is formed) at the central portion of the image sensor 28 via the lens 27.

On the other hand, in step S10, the control unit 41 causes the position detecting unit 117 to read the stage position. In step S11, the proximity limit position Dlimit is compared with the read stage position. More specifically, in step S10 (i.e., an example position detecting process), the position detecting unit 117 (see FIG. 1A) detects the position of the measurement unit 110 based on an alignment index image (whose virtual image is formed at the corneal focal position) in a state where the alignment has been completed.

If the operator operates the joystick 101 so erroneously that the measurement unit B reaches the proximity limit position (YES in step S11), then in step S12, the control unit 41 stops the Z-axis driving motor 108 and displays a caution message for the operator, for example, on the LCD monitor 116. Then, the operation returns to step S9 to perform the alignment again.

As described above, after the arithmetic processing in steps S6 and S7 has been completed, in steps S10 to S12 (i.e., an example control process), the operator controls the measurement unit 110 in such a way as to set the proximity limit position for the measurement unit 110 to be spaced a predetermined distance from the apex of the cornea of the subject's eye E in the working distance direction, irrespective of the radius of curvature of the cornea of subject's eye E.

In step S13, namely after the positioning (alignment) has been completed, it is determined whether the operator has pressed the measurement switch provided on the joystick 101 to start the intraocular pressure measurement. In step S14, the operator performs the intraocular pressure measurement a predetermined number of times. If the intraocular pressure measurement has been completed, then in step S15, the operator terminates the sequential measurement. More specifically, in steps S13 and S14 (i.e., an example ophthalmologic information acquisition process), the apparatus sets a movable range in the working distance direction so as not to exceed the proximity limit position relative to the subject's eye E. The apparatus acquires information about the intraocular pressure of the subject's eye E at a predetermined position within the movable range.

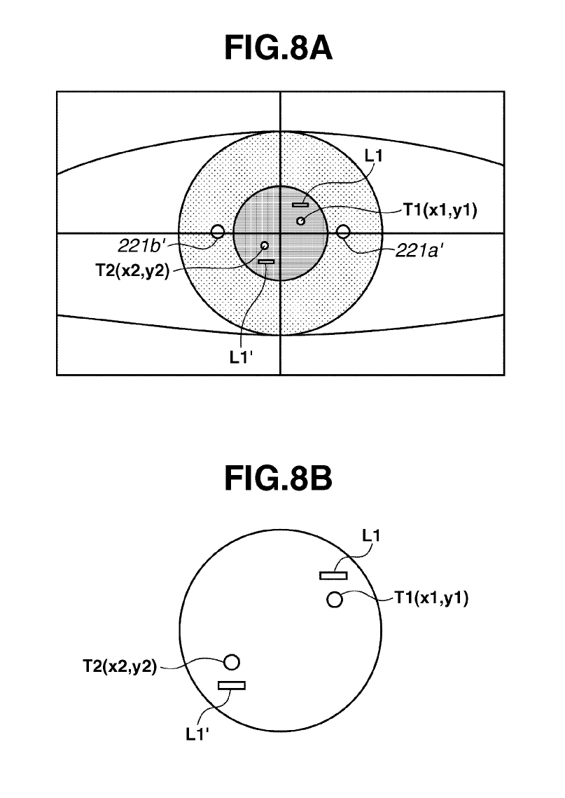

In the present exemplary embodiment, in step S9, it is useful to display a bright spot image position (i.e., a corneal reflection image position) converted from the proximity limit position determined in step S7 through separation and deflection by the prism 26 (i.e., a light flux deflection unit). More specifically, as illustrated in FIGS. 8A and 8B, it is useful to display a corneal reflection image that indicates the positioning state in the working distance direction together with predetermined marks each indicating the proximity limit (e.g., characters indicated by lines L1 and L1′ in the drawings) on the LCD monitor 116 to let the operator know the proximity limit.

According to the present exemplary embodiment, it is feasible to maintain a constant distance between the cornea of a subject's eye and the proximity limit position for a unit configured to acquire information about the intraocular pressure of the subject's eye, irrespective of the radius of curvature of the cornea of the subject's eye. Therefore, it is feasible to prevent the subject from having awful feeling when the apparatus is approaching the subject's eye even if the subject's eye has a larger curvature radius. Further, the operator can easily perform a positioning operation because the subject does not move the eye due to awful feeling. The operator can quickly and stably acquire the information about the intraocular pressure. Further, as the apparatus performs setting of the proximity limit position, it is unnecessary for the operator to set the limit position before starting the intraocular pressure measurement.

Further, the proximity limit position can be displayed on the display unit. Therefore, the operator can visually confirm the proximity limit in the working distance direction. Thus, the operability can be improved.

### Modification Example 1

In the above-mentioned exemplary embodiment, Dlimit=Dpos−ΔD−Do (constant value) is derived on the premise that Dr−Dt+Dnear=Do is a constant value because Dr−Dt is a constant value and Dnear is a setting value. However, the present invention is not limited to the above-mentioned example. More specifically, the present invention is employable even in a case where Dr−Dt is not a constant value (i.e., in a case where the tonometer position is variable in relation to the refractometer/keratometer position). In this case, a control unit 84 controls the proximity limit position based on an output of a position detecting unit 82 configured to detect the positioning completed position in the working distance direction and an output of a unit 83 configured to acquire information about the radius of curvature of the cornea, and further based on the working distance values Dr and Dt or the difference (Dr−Dt) of these values.

### Modification Example 2

The ophthalmologic apparatus described in the above-mentioned exemplary embodiment includes the refractometer/keratometer (i.e., an ophthalmologic multifunction peripheral) and the tonometer, which are integrally accommodated in a single casing. However, the ophthalmologic apparatus can be configured to include a keratometer (i.e., an ophthalmologic multifunction peripheral) and a tonometer, which are integrally accommodated in a single casing.

Further, the present invention is applicable to a tonometer (i.e., a single-function ophthalmologic apparatus) that is configured to acquire information about the radius of curvature of the cornea of the subject's eye E from a storage unit (e.g., a medical IC card) in which the information is stored beforehand.

Further, the present invention can be realized by performing the following processing according to an ophthalmologic control program. More specifically, the processing includes supplying a software program that can realize the functions of the above-mentioned exemplary embodiments to a system or an apparatus via a network or an appropriate storage medium and causing a computer (or a CPU or a micro-processing unit (MPU)) of the system or the apparatus to read and execute the program.

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment (s) of the present invention, and by a method performed by the computer of the system or apparatus by, for example, reading out and executing the computer executable instructions from the storage medium to perform the functions of one or more of the above-described embodiment (s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-230782 filed Oct. 18, 2012, which is hereby incorporated by reference herein in its entirety.

What is claimed is:

1. An ophthalmologic apparatus comprising:
a positioning unit configured to perform positioning for a subject's eye and an apparatus body by projecting an index light flux on a cornea of the subject's eye and capturing a corneal reflection image with light reflected from the cornea of the subject's eye;
a position detecting unit configured to detect the position of the apparatus body when the positioning unit completes the positioning in a working distance direction;
a first acquisition unit provided on the apparatus body and configured to acquire information about a radius of curvature of the cornea of the subject's eye;
a second acquisition unit provided on the apparatus body and configured to acquire information about an intraocular pressure of the subject's eye at a predetermined position in a movable range, which is set in the working distance direction in such a way as not to exceed a proximity limit position relative to the subject's eye; and
a control unit configured to control the second acquisition unit based on outputs of the position detecting unit and the first acquisition unit in such a way as to maintain a predetermined distance between the cornea of the subject's eye and the proximity limit position for the second acquisition unit in the working distance direction, regardless of the radius of curvature of the cornea of the subject's eye.

2. The ophthalmologic apparatus according to claim 1, wherein the proximity limit position is a position where a front end of a nozzle that acquires information about the intraocular pressure of the subject's eye is closest to the subject's eye.

3. The ophthalmologic apparatus according to claim 1, further comprising an acquisition unit configured to acquire eye refractivity/cornea shape of the subject's eye.

4. The ophthalmologic apparatus according to claim 3, wherein the control unit is configured to obtain a correction amount that is equivalent to a half of a difference between a curvature radius of the subject's eye and a curvature radius of a standard subject's eye and to set a distance obtained by adding the correction amount to a standard proximity distance predetermined for the standard subject's eye as a proximity distance corresponding to the proximity limit position relative to the subject's eye, and is configured to control the second acquisition unit based on the output of the position detecting unit in such a way as to prevent the second acquisition unit from approaching the subject's eye beyond the proximity distance.

5. The ophthalmologic apparatus according to claim 4, wherein the control unit is configured to determine the proximity distance corresponding to the proximity limit position using the following formula:
Dlimit=Dpos−(Dr−Dt+Dnear+ΔD)
where Dlimit represents the proximity limit position, Dpos represents the position of the apparatus body in an eye refractivity/cornea shape measurement, Dr represents a working distance of the apparatus body from the apex of the cornea in the eye refractivity/cornea shape measurement, Dt represents a working distance of the apparatus body from the apex of the cornea in an intraocular pressure measurement, Dnear represents a standard proximity distance relative to the standard subject's eye, and ΔD represents the half of the difference between the curvature radius of the subject's eye and the curvature radius of the standard subject's eye.

6. The ophthalmologic apparatus according to claim 1, further comprising a display unit configured to display the corneal reflection image that indicates a positioning state in the working distance direction and a mark that indicates the proximity limit position.

7. The ophthalmologic apparatus according to claim 6, wherein the positioning unit includes a light flux deflection unit configured to separate and deflect the corneal reflection image,
wherein the control unit is configured to convert the mark into a corneal reflection image position and to cause the display unit to display the corneal reflection image separated by the light flux deflection unit together with an anterior eye image of the subject's eye.

8. The ophthalmologic apparatus according to claim 1, further comprising a storage unit storing a table that associates the acquired information about the radius of curvature of the cornea with a correction amount for the proximity limit position in the working distance direction,
wherein the control unit is configured to control the proximity limit position while referring to the table.

9. An ophthalmologic control method comprising:
performing positioning for a subject's eye and an apparatus body by projecting an index light flux on a cornea of the subject's eye and capturing a corneal reflection image with light reflected from the cornea of the subject's eye;
detecting the position of the apparatus body when the positioning in a working distance direction is completed;
causing a first acquisition unit provided on the apparatus body to acquire information about a radius of curvature of the cornea of the subject's eye;
controlling a second acquisition unit provided on the apparatus body and configured to acquire information about an intraocular pressure of the subject's eye based on the detected position of the apparatus body and the acquired information about the radius of curvature of the cornea, in such a way as to set the proximity limit position in relation to the subject's eye to be a predetermined distance away from the cornea of the subject's eye in the working distance direction, regardless of the radius of curvature of the cornea of the subject's eye; and
causing the second acquisition unit to acquire information about the intraocular pressure of the subject's eye at a predetermined position in a movable range, which is set in the working distance direction in such a way as not to exceed the proximity limit position.

10. A computer-readable storage medium storing a program that causes a computer to perform the ophthalmologic control method according to claim 9.

11. An ophthalmologic apparatus comprising:
a first acquisition unit configured to acquire information about a radius of curvature of a cornea of a subject's eye;
a second acquisition unit configured to acquire information about an intraocular pressure of the subject's eye; and
a determination unit configured to determine a proximity limit position of the second acquisition unit relative to the subject's eye in a working distance direction based on the acquired information about the radius of curvature of the cornea.

12. An ophthalmologic control method comprising:
acquiring information about a radius of curvature of a cornea of a subject's eye;
acquiring information about an intraocular pressure of the subject's eye; and
determining a proximity limit position of a second acquisition unit, which acquires information about an intraocular pressure of the subject's eye, relative to the subject's eye in a working distance direction based on the acquired information about the radius of curvature of the cornea.

13. A computer-readable storage medium storing a program that causes a computer to perform the ophthalmologic control method according to claim 12.
